(19) United States
(12) Patent Application Publication
(10) Pub. No.: US 2019/0223864 A1
(43) Pub. Date: Jul. 25, 2019

(54) SURGICAL RETRACTOR DEVICE TO PROTECT UNDERLYING TISSUE

(71) Applicant: University of Saskatchewan, Saskatoon (CA)

(72) Inventor: Yigang Luo, Saskatoon (CA)

(21) Appl. No.: 16/331,224

(22) PCT Filed: Oct. 16, 2017
(86) PCT No.: PCT/CA2017/051225

§ 371 Date: Mar. 7, 2019

Related U.S. Application Data
(60) Provisional application No. 62/408,473, filed on Oct. 14, 2016.

(51) Int. Cl. A61B 17/04; A61B 90/00

(52) U.S. Cl. CPC A61B 17/0493; A61B 90/08; A61B 2017/0212; A61B 2090/08021; A61B 17/0483

(57) ABSTRACT

A surgical retractor device holds back underlying tissue from a tissue wall when suturing through the tissue wall with a suture passer. The retractor device comprises a barrier material having an upper barrier surface for spanning between the tissue wall and the underlying tissue and which is resistant to being punctured by the suture passer. The barrier material is operable between a working position in which the upper barrier surface occupies a first prescribed area and can be positioned between the tissue wall and the underlying tissue, and a collapsed position in which the barrier material is manipulated relative to the working position such that the upper barrier surface occupies a second prescribed area that is less than the first prescribed area such that the barrier material can pass through an incision in the tissue wall.

### FIELD OF THE INVENTION

The present invention relates to a surgical retractor device for insertion between a tissue wall and an underlying tissue to hold back the underlying tissue from the tissue wall when suturing through the tissue wall using a suture passer, for example to suture a surgical mesh to the underside of the tissue wall. More particularly, the present invention relates to a surgical retractor device which functions as a shield to protect the underlying tissue by being resist to puncturing by the suture passer.

### BACKGROUND

Massive ventral hernia is not an uncommon condition in adults. Generally, if the hernia measures >15 cm on the long axis laparoscopic surgery becomes challenging and surgeons often opt for incisional or open repair (1). Successful repair procedures involves the application of a mesh underneath the abdominal wall to enhance the repair. The procedure often requires a suture passer as shown in FIG. 1, which is a sharp object that passes through the abdominal wall, brings the sutures attached to the mesh out of the abdomen and anchors the mesh underneath the abdominal wall. One apparent risk in this procedure is that the suture passer can incidentally puncture the underlying intestines. To address this issue, surgeons use a spatula device (for example a thin sheet of metal as shown in FIG. 1) underneath the abdominal wall to stop the suture passer once it completely passes the abdominal wall. Not only is this cumbersome, but the surgeon assistant must accurately estimate the location of where the suture passer will come through and place the spatula device accordingly for every anchoring suture. Therefore, the surgery proceeds slowly, with no guarantee of abdominal protection. A more practical and safe solution that does not require extra assistance is needed.

The following references, referred to by number in this document, are herein incorporated by reference.
1. Dehn T. Incisional Hernia Repair—Laparoscopic or Open Surgery? Ann R Coll Surg Engl. 2009 November; 91(8):631-6.
2. Oderich G S, Panneton J M, Hofer J, Bower T C, Cherry K J, Sullivan T, et al. Iatrogenic operative injuries of abdominal and pelvic veins: a potentially lethal complication. J Vasc Surg. 2004 May; 39(5):931-6.
3. Falconi M, Pederzoli P. The relevance of gastrointestinal fistulae in clinical practice: a review. Gut. 2001 Dec. 1; 49(Supplement 4): iv2-iv10.
4. Okabayashi K, Ashrafian H, Zacharakis E, Hasegawa H, Kitagawa Y, Athanasiou T, et al. Adhesions after abdominal surgery: a systematic review of the incidence, distribution and severity. Surg Today. 2014 March; 44(3):405-420.
5. Kingsnorth A: The management of incisional hernia. Ann R Coll Surg Engl 2006; 88:252
6. Leppaniemi A, Tukiainen E: Reconstruction of complex abdominal wall defects. Scandinavian J Surgery. 2013; 102: 14.
7. Leparjemi A, Tukjainen E: Planned hernia repair and late abdominal wall reconstruction. World J Surg. 2012; 36:511
8. Andersen, L P H, Gogenur M K I and Rosenberg J: Long-term recurrence and complication rates after incisional hernia repair with the open onlay techniques. BMC Surgery 2009, 9:6 doi: 10.1186/1471-2482-9-6
9. Vorst A L, Kaoutzanis C, Carbonell A M, Franz M G. Evolution and advances in laparoscopic ventral and incisional hernia repair. World journal of gastrointestinal surgery. 2015; 7(11):293-305.
10. Rosen M J. Polyester-based mesh for ventral hernia repair: is it safe? American journal of surgery. 2009; 197(3):353-9.
11. Choi J J, Palaniappa N C, Dallas K B, Rudich T B, Colon M J, Divino C M. Use of mesh during ventral hernia repair in clean-contaminated and contaminated cases: outcomes of 33,832 cases. Annals of surgery. 2012; 255(1):176-80.
12. Zhang Z, Bernard E, Luo Y, et al: Animal models in xenotransplantation. Expert Opinion Invest. Drugs. 9(9): 2051-2068
13. Hannon J P, et al. Normal Physiological Values for Conscious Pigs used in Biomedical Research. Letterman Army Institute of Research, Presidio of San Francisco, Calif. 94129-6800, USA. May 1989
14. Calisto J L, Kate V, et al. Laparoscopic Incisional Hernia Repair. Medscape Oct. 16, 2015
15. Read R C, Yoder G. Recent trends in the management of incisional herniation. Arch Surg. 1989. 124:485-488.

### SUMMARY OF THE INVENTION

According to one aspect of the invention there is provided a surgical retractor device for use when suturing through a tissue wall to hold back underlying tissue from the tissue wall, the device comprising:

a barrier material having an upper barrier surface for spanning between the tissue wall and the underlying tissue and which is resistant to being punctured by the suture passer;

the barrier material being operable between a working position in which the upper barrier surface occupies a first prescribed area, and a collapsed position in which the barrier material is manipulated relative to the working position such that the upper barrier surface occupies a second prescribed area that is less than the first prescribed area.

During mesh application in massive ventral hernia repair, the surgical retractor device described herein is easily applicable, flexible, foldable and puncture-proof to better facilitate the surgery as compared to the currently available spatula device.

Preferably a minimum dimension across the upper barrier surface of the barrier material between opposing edges of the upper barrier surface is reduced in the collapsed position relative to the working position.

In the illustrated embodiment, the barrier material comprises a plurality of barrier panels which overlap one another so as to collectively define the upper barrier surface. Preferably the barrier panels are movable relative to one another between the working position and the collapsed position in a spanning direction of the upper barrier surface.

In the preferred embodiment, the barrier panels are pivotal relative to one another between the working position and the collapsed position about a common pivot axis. When each barrier panel is elongate in a respective longitudinal direction between a first end and a second end, preferably the common pivot axis is longitudinally offset towards the first end of each barrier panel.

The device may further include a guide arranged to locate the panels relative to one another in the working position, for example by use of stops which engage between adjacent panels in the working position, or by use of gears which are operatively connected between the panels to deploy adjacent panels relative to one another uniformly from the collapsed position to the working position. In this manner, the guide may define a first limit which prevents movement of the panels beyond the working position when displacing the panels from the collapsed position towards the working position and/or a second limit which prevents movement of the panels beyond the collapsed position when displacing the panels from the working position towards the collapsed position.

The barrier panels are preferably formed of a barrier material which comprises flexible, plastic sheets of sufficient thickness to resist puncturing by the suture passer.

According to a second aspect of the present invention there is provided a method of preparing to suture through a tissue wall having an incision therein and an underlying tissue beneath the tissue wall using a suture passer, the method comprising:

providing a retractor device comprising a barrier material having an upper barrier surface which is resistant to being punctured by the suture passer, the barrier material being operable between a working position and a collapsed position, wherein:
in the working position, the upper barrier surface occupies a first prescribed area, a minimum dimension across the upper barrier surface of the barrier material between opposing edges of the upper barrier surface is greater than a maximum dimension of the incision in the tissue wall, the barrier material is receivable between the tissue wall and the underlying tissue, and the retractor device cannot pass through the incision, and
in the collapsed position, the barrier material is manipulated relative to the working position such that the upper barrier surface occupies a second prescribed area that is less than the first prescribed area, a minimum dimension across the upper barrier surface of the barrier material between opposing edges of the barrier surface is less than the maximum dimension of the incision, and the retractor device can pass through the incision; and

(b) collapsing the retractor device from the working position to the collapsed position such that the retractor device is capable of being inserted through the incision.

According to another aspect of the present invention there is provided a method of suturing through a tissue wall having an underlying tissue beneath the tissue wall using a suture passer, the method comprising:

providing a retractor device comprising a barrier material having an upper barrier surface which is resistant to being punctured by the suture passer, the barrier material being operable between a working position in which the upper barrier surface occupies a first prescribed area, and a collapsed position in which the barrier material is manipulated relative to the working position such that the upper barrier surface occupies a second prescribed area that is less than the first prescribed area;

inserting the retractor device in the collapsed position through an incision in the tissue wall;

deploying the retractor device from the collapsed position to the working position;

placing sutures through the tissue wall using the suture passer;

collapsing the retractor device from the working position to the collapsed position;

withdrawing the retractor device through the incision; and

tying the sutures.

In an embodiment, the method comprises suturing a surgical mesh beneath the tissue wall by placing the sutures through the tissue wall and through the surgical mesh using the suture passer. In a further embodiment, the tissue wall is an abdominal tissue wall.

### BRIEF DESCRIPTION OF THE DRAWINGS

One embodiment of the invention will now be described in conjunction with the accompanying drawings in which:

FIG. 1 is a photograph of two examples of a currently available suture passer.

FIG. 2 is a photograph of a currently available spatula device used for mesh application in massive ventral hernia repair surgery;

FIG. 3 is a schematic representation of the surgical retractor device shown in the working position;

FIG. 4 is a photograph of one model of foldable leaf-shaped surgical retractor device shown in the collapsed position;

FIG. 5 is a photograph of an initial setup of the makeshift abdomen, comprising of cardboard box with an air-pillow, designed to emulate the abdomen in which a material of the surgical retractor device was cut to the appropriate size of the box and placed between the lid and the air-filled sac;

FIG. 6 is a photograph of the post-experiment results with the airpillow intact and the lid with 42 poke sites made by the suture passer;

FIG. 7 is a photograph of the initial concept model of foldable leaf-shaped surgical retractor device shown in the collapsed position;

FIG. 8 is a photograph of the makeshift abdomen, comprising of cardboard box with an air-pillow, under testing with a suture passer;

FIG. 9 is a photograph of a pig abdominal surgery showing the surgical retractor device in place after some manual manipulation.

FIG. 10 is a photograph of a pig abdominal surgery showing the suture passer traversing the abdominal wall and coming into contact with the surgical retractor device in which the surgical retractor device successfully prevented the suture passer from injuring the underlying abdominal contents;

FIG. 11 is a photograph of a conclusion of abdominal surgery in which the sutures were used to anchor the mesh to the inside of the abdomen wall;

FIG. 12 is a set upper photographs showing a CT scan showing bowel obstruction and recurrent massive ventral hernia; a set of bottom left photographs showing operative findings, adhesion band, causing complete obstruction, and a set of bottom right photographs showing early postoperative and one month after the surgery;

FIG. 13 shows a different patient, 6 months after massive ventral hernia repair with a 27 cm×34 cm complex mesh using the anchoring technique described herein;

FIG. 14 is a plan view of a solidworks model of the retractor device;

FIG. 15 is a schematic representation of the model of FIG. 14 when force was applied;

FIG. 16 is a schematic representation of the stress result on one panel of the retractor device;

FIG. 17 is a schematic representation of the displacement result on one panel of the retractor device; and

FIG. 18 is a schematic representation of the strain result on one panel of the retractor device.

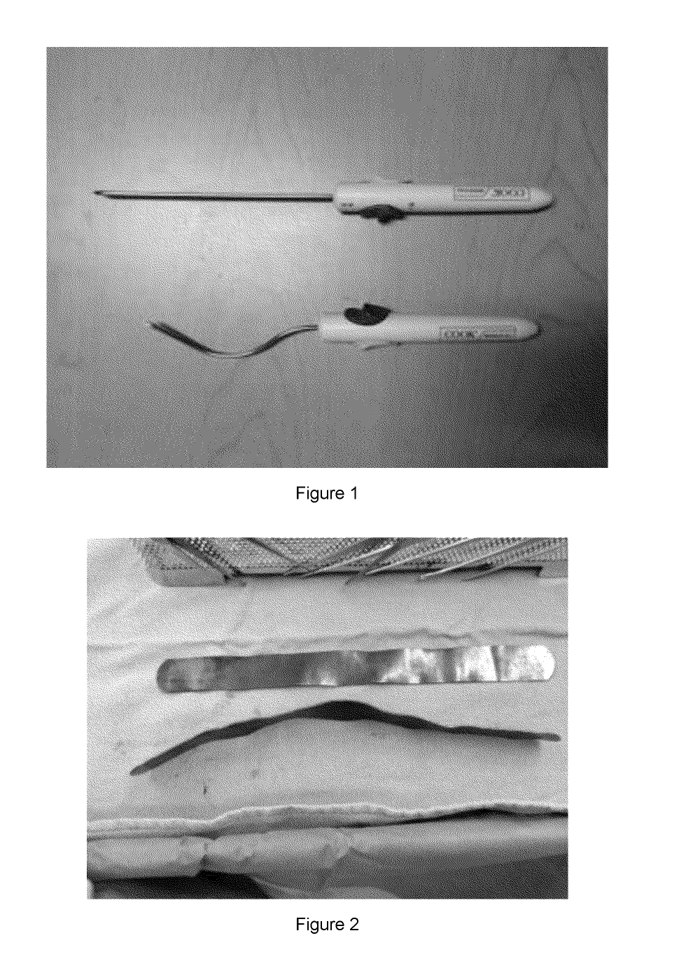

In the drawings like characters of reference indicate corresponding parts in the different figures.

### DETAILED DESCRIPTION

Referring to the accompanying figures, there is illustrated a surgical retractor device generally indicated by reference numeral 10.

The device 10 is particularly suited for use when suturing through a tissue wall, for example an abdominal wall, to protect underlying tissue from damage. In some instances, suturing through the tissue wall further comprises suturing through a surgical support mesh to affix the mesh to the underside of the tissue wall using a suture passer. The surgical retractor device sufficiently holds back underlying tissue (for example internal organs and the like) from the tissue wall to avoid puncturing of the underlying tissue by the suture passer during suturing by forming a barrier between the tissue wall and the underlying tissue which is resistant to being punctured by the suture passer.

The surgical retractor device 10 comprises a plurality of barrier panels 12. Each barrier panel 12 is a flat, flexible, plastic sheet of barrier material which is resistant to being punctured by the suture passer. The barrier panels are all identical to one another such that each panel is elongate in a respective longitudinal direction from a first end 14 to an opposing second end 16. The barrier panels are all connected to one another at a common pivot axis 18 which is positioned so as to be offset longitudinally towards the first end 14 of each barrier panel. The common pivot axis 18 is perpendicular to the panels. The barrier panels are pivotal relative to one another between a collapsed position and a working position.

In the collapsed position, the individual panels are all stacked in series with one another in the direction of the common pivot axis such that the uppermost one of the barrier panels fully defines an upper barrier surface of the collective panels of barrier material. More particularly the perimeter edge of the uppermost one of the panels defines the overall perimeter of the stacked panels, as well as defining the prescribed area that the barrier panels occupy. The width of panels defines the minimum dimension across the upper barrier surface of the barrier material between opposing edges of the upper barrier surface, which is less than the maximum dimension of the incision so that the retractor device can pass through the incision.

In the working position, each barrier panel extends radially outward from the common pivot axis from the first end to the second end of the respective barrier panel in a respective radial direction. The second ends 16 of the barrier panels 12 are evenly spaced apart from one another in a circumferential direction about the common pivot axis 18. The resulting upper barrier surface of the collective panels of barrier material in this instance is defined by a respective upper surface portion of each barrier panel. Furthermore, the perimeter edge of the resulting upper barrier surface defined by the barrier panels is formed by the plurality of panels collectively such that the second end of each barrier panel defines a respective portion of the overall perimeter edge of the resulting upper barrier surface of the barrier material. The upper barrier surface defined collectively by the barrier panels spans a first prescribed area which is more than double in size a second prescribed area defined by uppermost barrier panel in the collapsed position. Furthermore, a minimum dimension across the upper barrier surface of the barrier material between opposing edges of the upper barrier surface is reduced in the collapsed position relative to the working position. In this manner, the barrier member cannot pass through the incision in the working position but can pass through the incision in the collapsed position.

By pivotally coupling the barrier panels relative to one another for pivotal movement between the collapsed position and the working position thereof, the barrier panels move relative to one another generally in a direction of or within a generally common plane of the upper barrier surface.

To evenly position the barrier panels relative to one another in the circumferential direction about the common pivot axis 18 in the working position, additional guides may be provided which effectively position the panels relative to one another. For example, stops may be formed on the panels for engagement between adjacent ones of the panels in the working position when adjacent ones of the panels are adequately spaced apart from one another in the circumferential direction corresponding to the desired positioning in the working position. Alternatively, gears may be operatively connected between adjacent ones of the panels to uniformly deploy the panels relative to one another from the collapsed position to the working position. The stop may define a first limit of movement which prevents movement of the panels beyond the working position when displacing the panels from the collapsed position towards the working position, and/or a second limit of movement which prevents movement of the panels beyond the collapsed position when displacing the panels from the working position towards the collapsed position.

In yet further embodiments, the barrier panels may be interconnected by other means, for example by providing a track on one or more barrier panels which interlocks with a corresponding follower on one or more other panels to guide relative sliding between the barrier panels. In this instance, the barrier panels preferably remain slidable relative to one another within the direction that the upper barrier surface primarily spans so as to be well suited for deployment between the collapsed and working positions while being received between the tissue wall and the underlying tissue.

Although the barrier panels are shown to be flat and planar in the illustrated embodiment, in yet further embodiments the barrier panels may be non-planar; however, the barrier panels preferably remain low in profile by having a narrow thickness relative to dimensions between opposing side edges of the upper barrier surface so as to remain well suited for insertion between a tissue wall and underlying tissue.

In yet further embodiments, the barrier material may be defined by a single barrier panel which can be expanded from the collapsed position to the expanded position by flexing, folding, or stretching for example. In this instance, an underlying frame which can be extended by sliding or pivoting different frame portions relative to one another may support the barrier panel material thereon.

According to the present invention, the process of suturing a surgical mesh beneath a tissue wall having an underlying tissue beneath the tissue wall can be accomplished using a suture passer with less risk of puncturing the underlying tissue than when using conventional surgical tools. Initially, the retractor device is inserted through an incision in the tissue wall in the collapsed position. The retractor device is then deploying into the working position to allow placement of sutures between the surgical mesh and the tissue wall using the suture passer while shielding and holding back the underlying tissue. The retractor device can then be collapsed and withdrawn through the incision in the collapsed position. The incision can then be mostly or fully closed to properly position the mesh relative to the tissue wall and allow completion of the suture anchoring between the surgical mesh and the tissue wall.

### Experimental Development

Firstly, we conceived the shape, size, and method of expansion of the device by designing a surgical retractor device composed of several layers of leaf-shaped paper and cardboard material. Secondly, a makeshift abdominal model was created by placing air-filled bag in a small cardboard box. With a hard plastic sheet placed between the air-filled bag and the lid, 42 holes were made from the top using a suture passer. This experiment was performed twice to ensure that the selected plastic was puncture-proof. Thirdly, a novel surgical retractor device was finalized with 7 foldable leaflets of the hard plastic joined together by a string on one end. Finally, the new surgical retractor device was tested on mock pig abdominal massive hernia repair surgery (n=2).

### Description of Animal Study

Two healthy pigs (large white, 30-40 kg, females) from Prairie Swine Center, Saskatoon, Saskatchewan, Canada, were sacrificed for a separate kidney procurement and ex vivo perfusion study (Protocol #416438) in the operating room of Veterinary Medical Center at the College of Veterinary Medicine, University of Saskatchewan. The retractor designed in the present study was tested when the abdominal wall was sewn back together. An appropriately sized flexible plastic sheet (12 cm in diameter) was used in place of a mesh. We tested our retractor on both pigs regarding its manipulation (insertion, unfolding, refolding and retrieval), its protective reliability, and the time required in placement of a mesh.

### Description of Clinical Case

A 56 year old female, BMI (body mass index) 42, with recurrent massive ventral after five repairs before including mesh repair and abdominal wall component separation reconstruction, came to the emergency department with persistent bowel obstruction for one week. She was treated conservatively with the nasal gastric tube and IV nutrition support. However, her symptoms were not relieved. CT scan showed complete bowel obstruction with definitive transitional site in small bowel incarcerated in the recurrent massive ventral hernia in the upper abdomen. The abdominal wall defect was about 20 cm. She was taken to the operating room, where she had laparotomy, mesh repair in our novel technique but with a spatula (FIG. 12).

### Results

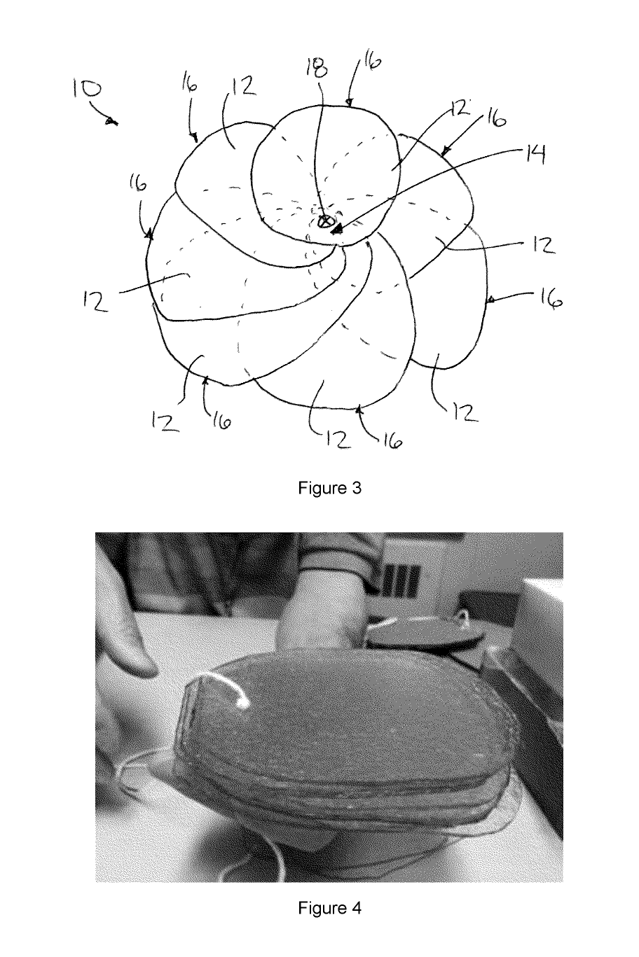

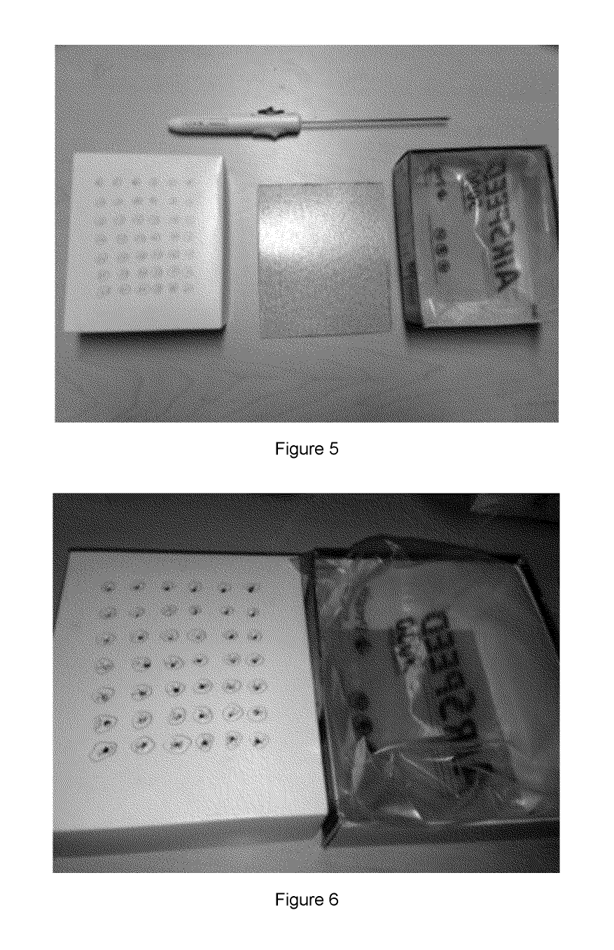

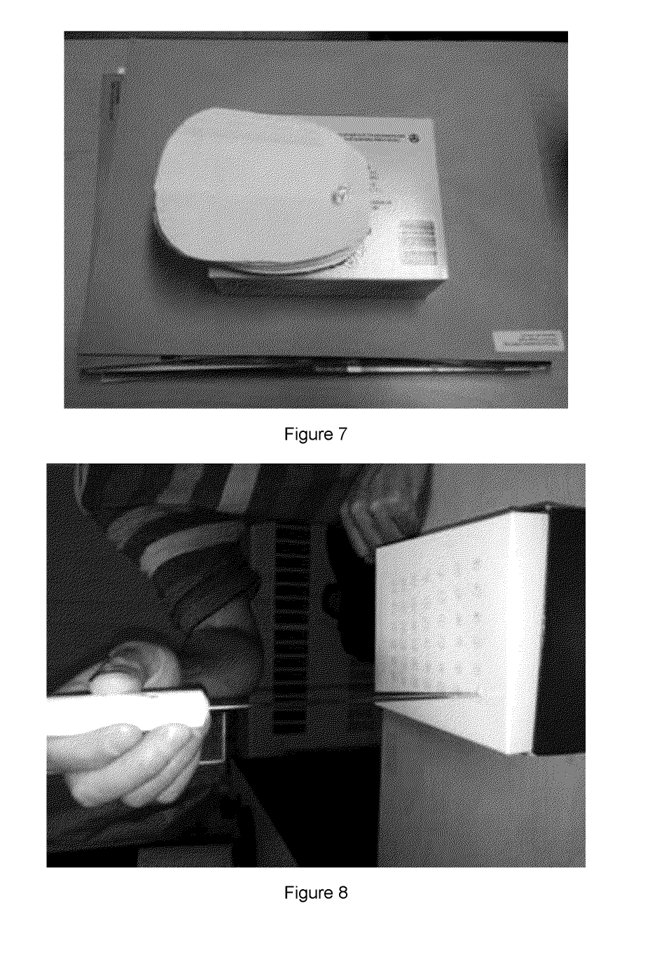

Simple and innovative retractor design for use in abdominal hernia repairs. Our concept model was made from partially overlapping layers of leaves in paper and cardboard. Appropriate size and shape, were reached together with unfolding and refolding capability as 9 cm×12 cm elliptical leaflets, according to our further application in animal trial, to be protective over 15-20 cm in diameter intraabdominal area. The strength of the plastic sheet against puncture due to the suture passer was tested on our makeshift abdomen. A total of 84 punctures were made, with no penetration, suggesting the plastic material was very effective in protection (FIGS. 5, 6). The next stage of our study involved making the device from the plastic material (Office Staples mat) and assembly. The final product consists of 7 leaflets of plastic material held together by a string in medial central side (FIG. 3, 4). The device was flexible yet strong and easy to fold and unfold. It could satisfactorily cover 15-20 cm intra-abdominal surgical area.

### Abdominal Surgery on Pig Model

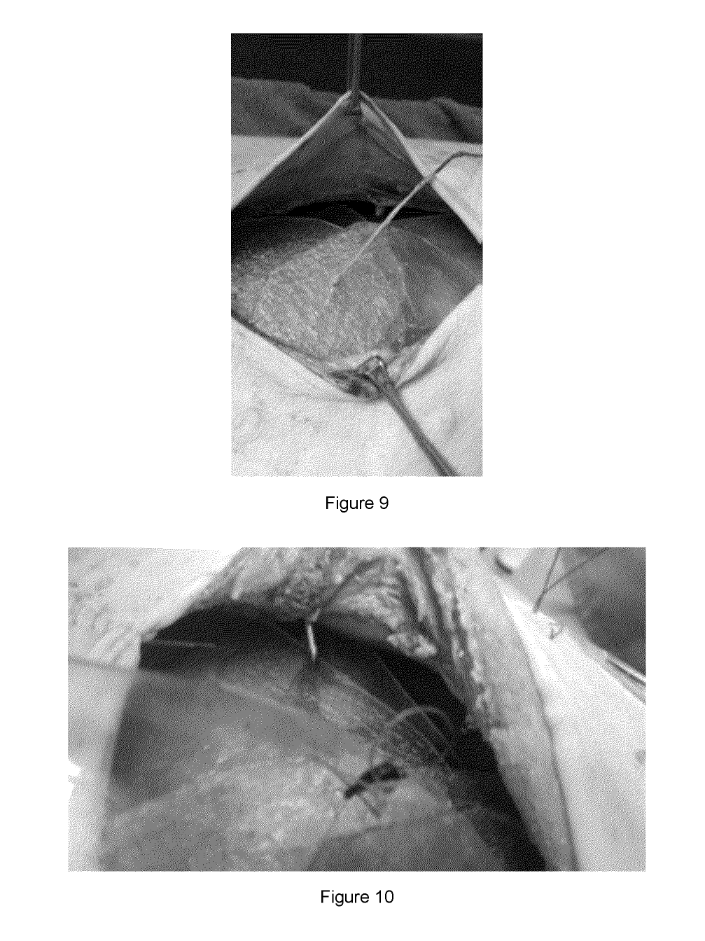

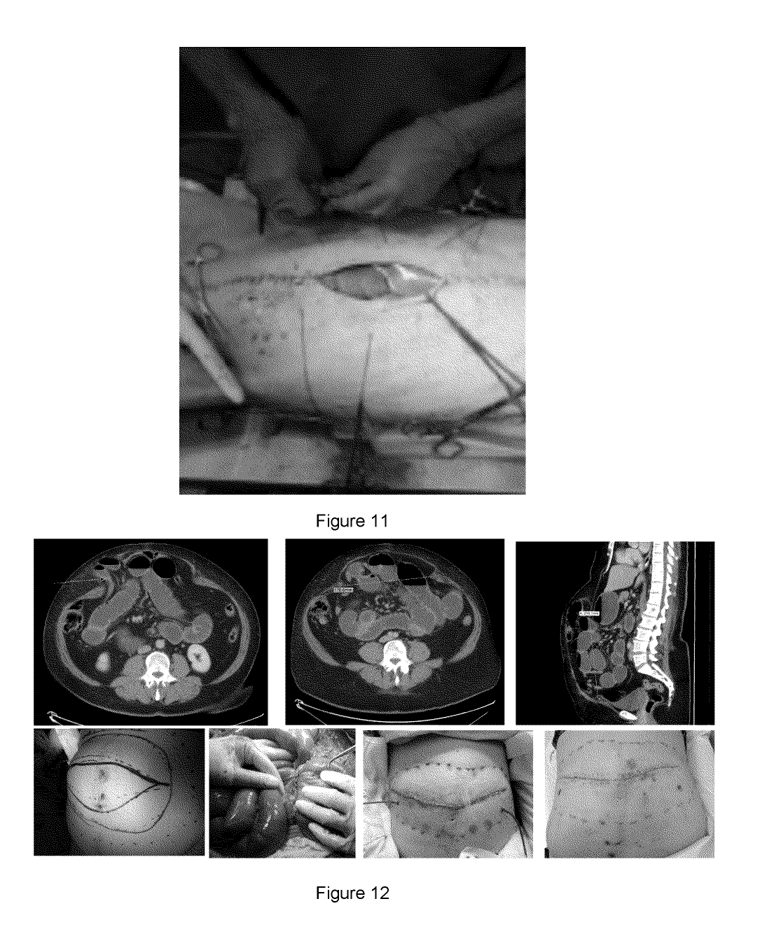

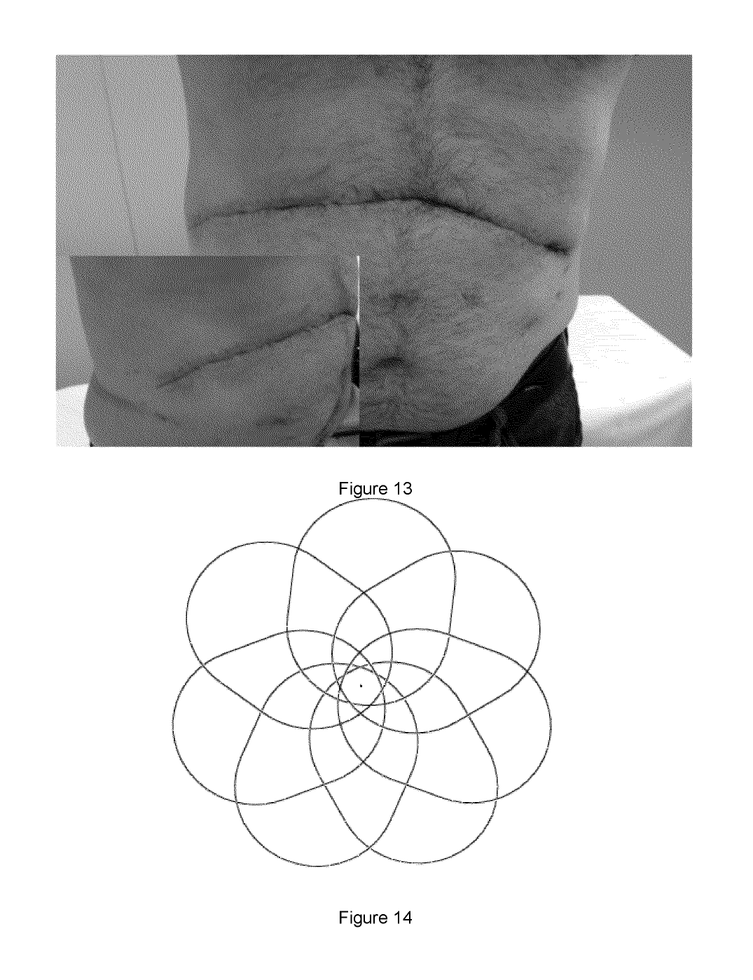

With the device pictured as shown in FIGS. 3, 4, our experiment with two sacrificed pigs showed that the retractor provided ample protection with no injury to the abdominal contents from the suture passer (FIGS. 9, 10). The surgeon noted that the device insertion into the abdomen required some manual manipulation to ensure that all areas of the abdomen were covered (FIG. 9). The removal process on the other hand was quick and the compact size of the folded retractor proved beneficial (FIG. 11). Only 26-28 minutes were needed to suture the mesh inside the abdomen. Roughly half the time was needed to do the same repair with currently available equipment (spatula) in the operating room.

### Clinical Case Result:

During the surgery, an adhesion band causing complete bowel obstruction was found. It was divided. Extensive adhesions were lysed and the obstruction was released. The hernia defect, 21 cm×18 cm, was repaired with a 25 cm×35 cm complex mesh using our new anchoring technique. She recovered very well after the surgery with no complications. Follow-up 3 months showed that she was doing well, back to work, with no complications or recurrence (FIGS. 12&13).

### Discussion

Massive ventral hernia happens commonly up to 30% after abdominal surgeries, especially after trauma laparotomy and multiple abdominal surgeries with postoperative incisional complications, such as infection, dehiscence, and so on [14]. These hernias often cause significant morbidity, leading to poor life quality and possible complication of bowel obstruction [5]. Repair of massive ventral hernia is frequently a challenge in general surgery, with up to more than a 30% failure rate, in addition to postoperative pain, incision complications, etc. [5]. Every year, some 100,000-150,000 ventral hernia were repaired in the United States [15]. Over the last 20 years, tension-free repair of massive ventral hernia with mesh, especially by laparoscopy, becomes gradually popular with much better outcomes, such as less postoperative abdominal pain and a decreased recurrence rate close to 15% [5-11, 14]. However, most research on massive ventral hernia repair is about modification of the existing approaches or surgical methods, including abdominal wall reconstruction. There was little research on surgical instruments or devices for this purpose, in particular, design of a retractor to facilitate this type of surgery. Inlay mesh repair is the choice of surgical techniques, where the mesh is inserted and attached to inside surface of abdominal wall. Although the laparoscopic approach is to facilitate inlay mesh placement with less likelihood of infection, seroma or hematoma, it carries high rate of incidental enterotomy and usually takes longer surgical time [14]. In addition, very large ventral hernia, recurrent ventral hernia, incarcerated hernia and hernia with multiple past surgeries might be relative contraindicative for laparoscopic surgery [14]. On the other hand, open hernia repair usually needs to free the facia-muscular layer to facilitate the anchoring an inlay mesh and, thus creates a potential dead space between subcutaneous layer and facia-muscular layer. This approach leads to the shortcomings of open surgery: more postoperative pain, high rate of seroma, hematoma and mesh infection as well as longer patient stays in the hospital [14].

Our approach is to use an open technique but a laparoscopic instrument to speed up the surgery by not freeing the facia-muscular layer to omit the complications caused by the above mentioned dead space. After freeing the adhesions inside the abdomen, we repair the hernia by anchoring the inlay mesh with sutures across the whole abdominal wall with the help of laparoscopic suture passer. This technique has been used in clinical cases with good results. The patient hospital stay is shorter with little wound complications. However, incidental bowel perforation is a potential complication and although rare its consequence are devastating [2]. In order to prevent incidental injury to the intestines and other intra-abdominal organs intra-operatively, we usually have to use a spatula, which is relatively small, narrow and inadequate in providing complete protection to the intra-abdominal organs, including the intestines. We need to move and ensure the spatula stay precisely in place of every suture passer puncture, which not only slows down the surgery but is also less reliable. Therefore, we invented a new abdominal retractor, which is designed as being foldable and fan-shaped. It can protect all the intra-abdominal organs including the intestines from incidental suture passer injury.

### A Novel Retractor Device for Mesh Application

This preliminary concept study involved innovative designing, creation, and testing of a surgical tool that will assist surgeons in mesh anchoring during open hernia repair surgery. Our results demonstrated adequate protection, foldable design and easy application. There is evidence for further development of this simple useful device. It is important to note that our device does not require extra personnel to hold it in place, whereas the currently available spatula requires at least a two-person team. Reducing manpower, speeding up the surgery and prevention of complications will save tax-payers' money on the health system and save patients from suffering.

A gear mechanism that turns the leaflets spreading to a pre-set perfect overlap would be ideal. Re-usability of the device might be another consideration in our future study. Engineering optimization of leaflet shape, thickness, flexibility, number and biocompatibility is now undertaken.

An innovative, foldable and reliable abdominal retractor useful for a new surgical technique in massive ventral hernia repair was proved in concept. Further optimization and verification are in process.

Iatrogenic bowel perforation during abdominal surgery is a rare complication; however, its consequences are devastating and can lead to patient instability if not addressed immediately [2].

We are pleased with the strength, flexibility, and cost of the material and believe these factors make this device worthy of further testing. It is important to note here that our device does not require extra personnel to hold it in place, whereas the currently available spatula requires at least a two-person team.

Future work would include modifying the surgical retractor device such that once it is placed in the abdomen, little manual manipulation is needed to open it. A gear mechanism that turns the leafs to a “preset amount” and that provides significant overlap would be ideal. We would like to also examine the re-usability of the device and how many surgeries the device can survive before a replacement is necessary. Comparison studies with currently available spatula on live animal under sterile conditions is necessary for further evaluation of this novel surgical retractor device for safety, manipulation, surgical time, and post-operative complications, such as infection, fistula, adhesions, and recurrence of hernias [3, 4].

This study innovated the formulation and trials of a novel device that can be used during mesh installation for massive ventral hernia repair. We have successfully shown that a simple retractable device composed of leaf-shaped plastic sheets is a superior alternative to the currently available spatula device.

### Finite Element Analysis

The finite analysis is a numerical method for solving problems of engineering and mathematical physics. The method yields approximate values of the unknowns at discrete number of points over the domain. To solve the problem, it subdivides a large problem into smaller, simpler parts that are called finite elements (Finite element method. (2017 Apr. 10). Retrieved May 11, 2017, https://en.wikipedia.org/wiki/Finite_element_method). Since this retractor contains 7 pieces of blades, what we are going to do is to analysis one piece of blade.

We analysis the force applied into the surface of the retractor. The load required to pull the tissue to correct tension during suturing was varying between 1.5 and 2.5 N (mean 1.9 N) for skin (Frick, T., Marucci, D., Cartmill, J., Martin, C., & Walsh, W. (2001). Resistance forces acting on suture needles. Journal of Biomechanics, 34(10), 1335-1340. doi:10.1016/s0021-9290(01)00099-9). And we picked up a suture needle with the diameter D of 0.33 mm. FIG. 14 is the solidworks prototype of this retractor. In this study, we select alloy steel as the material.

The properties of alloy steel are listed in the following table:

| Property | Value | Units |
|---|---|---|
| Elastic Modulus | 2.1e−011 | N/m\hat{ }2 |
| Poisson's Ratio | 0.28 | N/A |
| Shear Modulus | 7.9e−010 | N/m\hat{ }2 |
| Mass Density | 7700 | kg/m\hat{ }3 |
| Tensile Strength | 723825600 | N/m\hat{ }2 |
| Compressive Strength | | N/m\hat{ }2 |
| Yield Strength | 620422000 | N/m\hat{ }2 |
| Thermal Expansion Coefficient | 1.3e−005 | /K |
| Thermal Conductivity | 50 | W/(m · K) |
| Specific Heat | 460 | J/(kg · K) |
| Material Damping Ratio | | N/A |

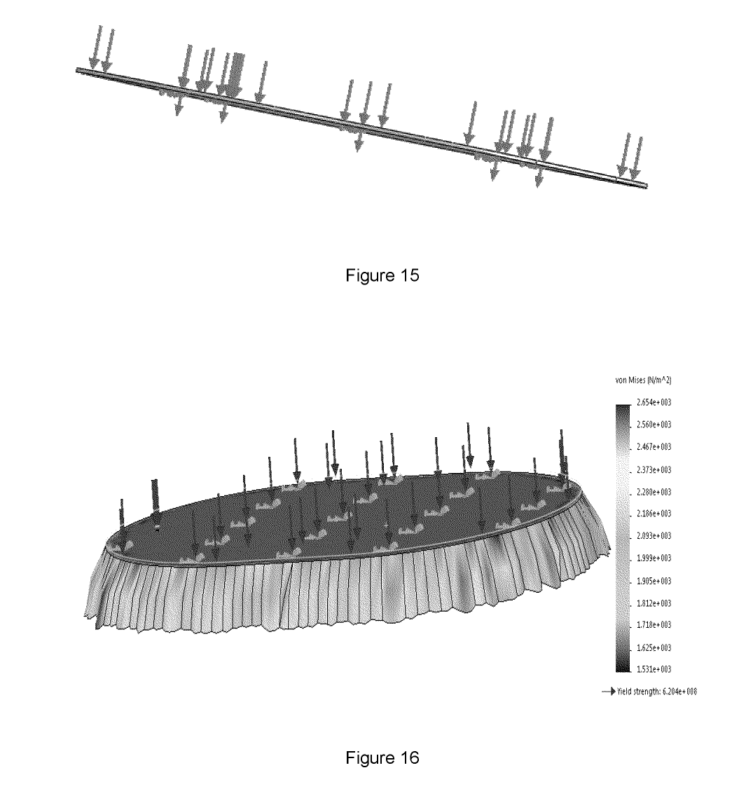

When 2.5N force was applied to one surface, the other surface was fixed (see FIG. 15), Which mimics the real situation during the surgery. One surface of the retractor is supported by the intestine, the force is applied by suture needle on the other side.

Here are the results when using Solidworks Simulation. FIG. 16 is the result about stress, as we can see, the maxima stress is 2.654e+003, whereas the yield strength is 6.204e+008. The stress is much lower than yield strength, which means it is not necessary to worry about the strength of the material. Meanwhile, the red area is concentrated around edge. To decrease any stress concentration, all sharp edges should be round (Bejgerowski, W., Gerdes, J. W., Gupta, S. K., & Bruck, H. A. (2011). Design and fabrication of miniature compliant hinges for multi-material compliant mechanisms. The International Journal of Advanced Manufacturing Technology, 57(5-8), 437-452. doi:10.1007/s00170-011-3301-y). This requires us to make the edge of blade round to avoid damaging the intestine.

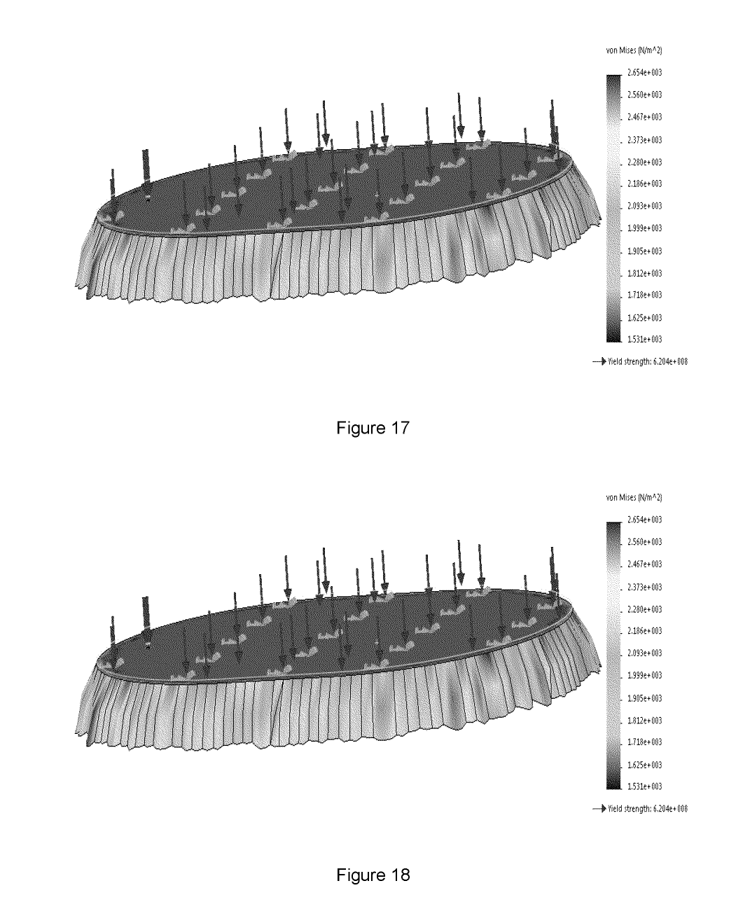

FIG. 17 illustrates the displacement when the force is applied. The maxima strain is minor when compared to original length, so it does not affect the structure. FIG. 18 shows the strain result. The Equivalent strain (ESTRN) is from 8.103e-009 to 1.133e-008. Therefore, it does not affect the structure of retractor either.

By Solidworks Simulation, we analyzed characteristics of every element including stress, displacement and strain. It is explicit to show readers and designers the results when the force is applied. And it helps how to select material and optimize the structure.

### Discussion

The purpose of the novel retractor is to keep suture needle from damaging intestine. Therefore, it is crucial to make sure the structure is strong enough without hurting intestine tissues. By FEA of solidworks simulation, we can find if it is good enough to be such a retractor. Every aspect we listed above satisfies the requirement, however, the material of alloy steel is not good enough because it is too hard. It is unavoidable to hurt intestine tissues. We are still looking for suitable material for this design. But the advantage of FEA is to determine if the suitable material is good enough to resist the force. At the same time, it helps designers to reduce the weight as well by changing the shape since the retractor is supported by intestine.

Since various modifications can be made in my invention as herein above described, and many apparently widely different embodiments of same made, it is intended that all matter contained in the accompanying specification shall be interpreted as illustrative only and not in a limiting sense.

1. A surgical retractor device for use with a suture passer when suturing through a tissue wall to hold back underlying tissue from the tissue wall, the device comprising:
a barrier material having an upper barrier surface for spanning between the tissue wall and the underlying tissue and which is resistant to being punctured by the suture passer;
the barrier material being operable between a working position in which the upper barrier surface occupies a first prescribed area, and a collapsed position in which the barrier material is manipulated relative to the working position such that the upper barrier surface occupies a second prescribed area that is less than the first prescribed area.

2. The device according to claim 1 wherein a minimum dimension across the upper barrier surface of the barrier material between opposing edges of the upper barrier surface is reduced in the collapsed position relative to the working position.

3. The device according to claim 1 wherein the barrier material comprises a plurality of barrier panels which partially overlap one another so as to collectively define the upper barrier surface.

4. The device according to claim 3 wherein the barrier panels are movable relative to one another between the working position and the collapsed position in a direction that the upper barrier surface spans.

5. The device according to claim 3 wherein the barrier panels are pivotal relative to one another between the working position and the collapsed position.

6. The device according to claim 5 wherein the barrier panels are pivotally connected relative to one another about a common pivot axis oriented generally perpendicularly to the barrier panels.

7. The device according to claim 6 wherein each barrier panel is elongate in a respective longitudinal direction between a first end and a second end and wherein the common pivot axis is longitudinally offset towards the first end of each barrier panel.

8. The device according to claim 3 further comprising a guide arranged to locate the panels relative to one another in the working position.

9. The device according to claim 8 wherein the guide defines a limit which prevents movement of the panels beyond the working position when displacing the panels from the collapsed position towards the working position.

10. The device according to claim 8 wherein the guide defines a limit which prevents movement of the panels beyond the collapsed position when displacing the panels from the working position towards the collapsed position.

11. The device according to claim 1 wherein the barrier material comprises a flexible sheet.

12. A method of preparing to suture through a tissue wall having an incision therein and an underlying tissue beneath the tissue wall using a suture passer, the method comprising:
providing a retractor device comprising a barrier material having an upper barrier surface which is resistant to being punctured by the suture passer, the barrier material being operable between a working position and a collapsed position, wherein:
in the working position, the upper barrier surface occupies a first prescribed area, a minimum dimension across the upper barrier surface of the barrier material between opposing edges of the upper barrier surface is greater than a maximum dimension of the incision in the tissue wall, the barrier material is receivable between the tissue wall and the underlying tissue, and the retractor device cannot pass through the incision, and
in the collapsed position, the barrier material is manipulated relative to the working position such that the upper barrier surface occupies a second prescribed area that is less than the first prescribed area, a minimum dimension across the upper barrier surface of the barrier material between opposing edges of the barrier surface is less than the maximum dimension of the incision, and the retractor device can pass through the incision; and
(b) collapsing the retractor device from the working position to the collapsed position such that the retractor device is capable of being inserted through the incision.

13. A method of suturing through a tissue wall having an underlying tissue beneath the tissue wall using a suture passer, the method comprising:
(a) providing a retractor device comprising a barrier material having an upper barrier surface which is resistant to being punctured by the suture passer, the barrier material being operable between a working position in which the upper barrier surface occupies a first prescribed area, and a collapsed position in which the barrier material is manipulated relative to the working position such that the upper barrier surface occupies a second prescribed area that is less than the first prescribed area;
(b) inserting the retractor device in the collapsed position through an incision in the tissue wall;
(c) deploying the retractor device from the collapsed position to the working position between the tissue wall and the underlying tissue;
(d) placing sutures through the tissue wall using the suture passer;
(e) collapsing the retractor device from the working position to the collapsed position;
(f) withdrawing the retractor device through the incision; and
(g) tying the sutures.

14. The method of claim 13, wherein the tissue wall is an abdominal tissue wall.

15. The method of claim 13, wherein step (d) further comprises suturing a surgical mesh beneath the tissue wall by placing the sutures through the tissue wall and through the surgical mesh using the suture passer.

16. The method of claim 15, wherein the tissue wall is an abdominal tissue wall.
